(19) United States
(12) Patent Application Publication
(10) Pub. No.: US 2011/0106134 A1
(43) Pub. Date: May 5, 2011

(54) Indwelling Temporary IVC Filter System with Aspiration

(75) Inventors: Dustin Thompson, Santa Rosa, CA (US); D.H. Perkins, Santa Rosa, CA (US)

(73) Assignee: Medtronic Vascular, Inc., Santa Rosa, CA (US)

(21) Appl. No.: 12/608,207

(22) Filed: Oct. 29, 2009

(51) Int. Cl. A61F 2/01; A61M 25/00

(52) U.S. Cl. 606/200; 604/510

(57) ABSTRACT

Indwelling temporary inferior vena cava filter systems are disclosed. Such filter systems provide for easy removal of the filter without the need for additional invasive procedures and provide for aspiration of captured emboli. Methods of using such systems for the capture and removal of emboli are described.

### FIELD OF THE INVENTION

The present invention relates to filters within a vessel. In particular, the present invention relates to vena cava filters that are indwelling and that are easily removable. More particularly, the present invention relates to a vena cava filter system that additionally employs means for removing trapped emboli.

### BACKGROUND OF THE INVENTION

Inferior Vena Cava (IVC) filters are currently used to prevent venous emboli from migrating through the heart to the lungs, resulting in a pulmonary embolism (PE). Pulmonary embolism is a blockage of the pulmonary artery by a blood clot, or thrombus. In ninety percent of cases, these blood clots originate in the lower extremity and travel through the inferior vena cava before passing through the right side of the heart and entering the lungs. In twenty-five percent of cases, the extent of the embolism leads to sudden death. Patients at risk for PE are trauma, surgical (e.g. hip replacement or spine repair), cancer, venous disease (including deep vein thrombosis and chronic venous insufficiency) patients. The current standard of care for PE patients, or patients at risk of a PE, is anticoagulation therapy. Patients contraindicated for anticoagulation therapy, due to bleeding risks, or those at high risk of an initial or recurrent PE are treated with an IVC filter. The devices are generally implanted within the IVC and function by capturing emboli contained in the blood stream before they can reach the lungs and cause permanent damage to the patient. They generally employ a series of legs or other features that are expanded in the vessel to form a conical-shaped filtering surface on which emboli are collected. To anchor the filter in the vessel and prevent it from migrating to the heart, hooks, barbs or similar piercing means on the legs are employed, making removal of the filter difficult. IVC filters implanted for an indefinite amount of time, deemed permanent, may also result in a permanent obstruction in the vessel and result in dangerous disruption of normal hemodynamic flow. Furthermore, the use of systemic anti-coagulant and/or thrombolytic agents presents additional risk to the patient due to the high systemic dosage of such agents that is needed to dissolve captured emboli. While IVC filters were originally designed as permanent implants, more recently there has been a focus on retrievable filters that can be removed once the threat of PE has passed. For example, an IVC filter need only be in place during and for a short time after certain medical and surgical procedures that carry a significant risk of PE. However, there are problems with retrievable IVC filters, including their endotheliazation and the aforementioned piercing means, which can make removal difficult and dangerous to the patient. In some cases, certain devices may result in the filter legs protruding into the vessel wall of the IVC by five or more millimeters. Retrievable IVC filters are not permanently attached to retrieval means and require a separate procedure to “snare” the filter to remove it from the patient. Another problem with retrievable IVC filters is how to safely remove captured emboli.

Accordingly, it would be desirable to have an IVC filter that can be implanted in a patient at risk for PE and is easily removable once the risk has passed without risk or damage to the patient, and where captured emboli are safely removed.

### SUMMARY OF THE INVENTION

The present invention relates to IVC filter systems that allow for temporary implantation of an IVC filter that is easily removed without the need for additional invasive procedures. The invention also relates to IVC filter systems having aspiration capability. The invention also relates to methods for capturing and removing venous emboli.

According to the teachings of the present invention there is provided an inferior vena cava filter system comprising a filter having proximal and distal ends, an elongate element having proximal and distal ends and an aspiration lumen therethrough, and a tubular enclosure surrounding said filter.

According to one representation of the present invention the elongate element and tubular enclosure form an integral structure in which they are longitudinally adjacent or concentric with each other and the filter comprises a shape memory metallic wire having a delivery configuration and a deployed configuration, wherein the delivery configuration is essentially straight and the deployed configuration is a conical coil shape.

According to another representation of the present invention the proximal end of the filter is attached to the distal end of the elongate element and the tubular enclosure is a sheath that is slidably disposed around the elongate element and filter.

According to a further feature of the present invention the proximal end of the elongate element contains an aspiration port.

According to a further feature of the present invention the proximal end of the elongate element contains an infusion port.

According to a further feature of the present invention the elongate element contains a guidewire lumen.

According to the teachings of the present invention there is provided a method of capturing and removing venous emboli by delivering the device to the inferior vena cava using the Seldinger technique. A guidewire to a target location in the inferior vena cava of a patient, delivering an inferior vena cava filter system over the guidewire to the target location, the system comprising a filter having proximal and distal ends, an elongate element having proximal and distal ends and an aspiration lumen therethrough, and a tubular enclosure surrounding said filter, wherein the proximal end of said elongate element further contains an aspiration port, wherein the filter comprises a shape memory metallic wire that is essentially straight when within the tubular enclosure and that assumes a conical coil configuration when deployed, delivering the filter to the target location, thereby allowing the filter to assume its shape memory conical coil configuration, leaving the filter in place for a determined period of time to capture emboli, aspirating captured emboli through the aspiration lumen, retracting the filter into the tubular enclosure, and removing the system from the patient.

According to further teachings of the present invention there is provided a method of capturing and removing venous emboli by delivering a guidewire to a target location in the inferior vena cava of a patient, delivering an inferior vena cava filter system over the guidewire to the target location, the system comprising a filter having proximal and distal ends, an elongate element having proximal and distal ends and an aspiration lumen therethrough, and a tubular enclosure surrounding said filter, wherein the proximal end of said filter is attached to the distal end of said elongate element and said tubular enclosure is a sheath that is slidably disposed around said elongate element and filter, sliding said sheath with respect to said elongate element to deploy the filter, leaving the filter in place for a determined period of time to capture emboli, aspirating captured emboli through the aspiration lumen, retracting the filter into the sheath, and removing the system from the patient.

According to a further feature of the present invention a pharmaceutical agent functioning as either an anti-coagulation agent and/or a thrombolytic agent is locally infused through an infusion port to assist in the prevention of emboli formation and/or dissolution of captured emboli, respectively.

### BRIEF DESCRIPTION OF THE DRAWINGS

FIG. 1 is a schematic view of an IVC filter system of the present invention

FIG. 2 is a schematic cut-away view of the distal portion of one embodiment of the IVC filter system of FIG. 1.

FIG. 3A is a view similar to FIG. 2, but without showing the elongate element structure.

FIG. 3B is a view similar to FIG. 3A, but with the filter deployed.

FIG. 4 is a perspective view of a filter according to one embodiment of the invention.

FIG. 5 is a radial cross-section of the device of FIG. 2, taken along lines 5-5.

FIG. 6A is a schematic cut-away view of the distal portion of another embodiment of the IVC filter system.

FIG. 6B is a view similar to FIG. 6A, but with the filter deployed.

FIG. 7 is a radial cross-section of the device of FIG. 6A, taken along lines 7-7, but with the addition of a guidewire lumen.

FIG. 8 is a perspective view of another filter embodiment that can be used instead of the filter shown with the device of FIGS. 6A and 6B.

FIG. 9 is a flowchart depicting one method of capturing and removing venous emboli.

FIG. 10 is a flowchart depicting another method of capturing and removing venous emboli.

### DETAILED DESCRIPTION OF THE INVENTION

The present invention relates to inferior vena cava (IVC) filter systems and methods. In particular, the present invention relates to IVC filter systems that allow for a filter to be temporarily implanted and easily removed without the need for a second catheterization procedure. The IVC filter system has aspiration capability for removal of emboli prior to removal of the device. The invention also relates to methods for utilizing the aforementioned IVC filter systems for the capture and removal of venous emboli.

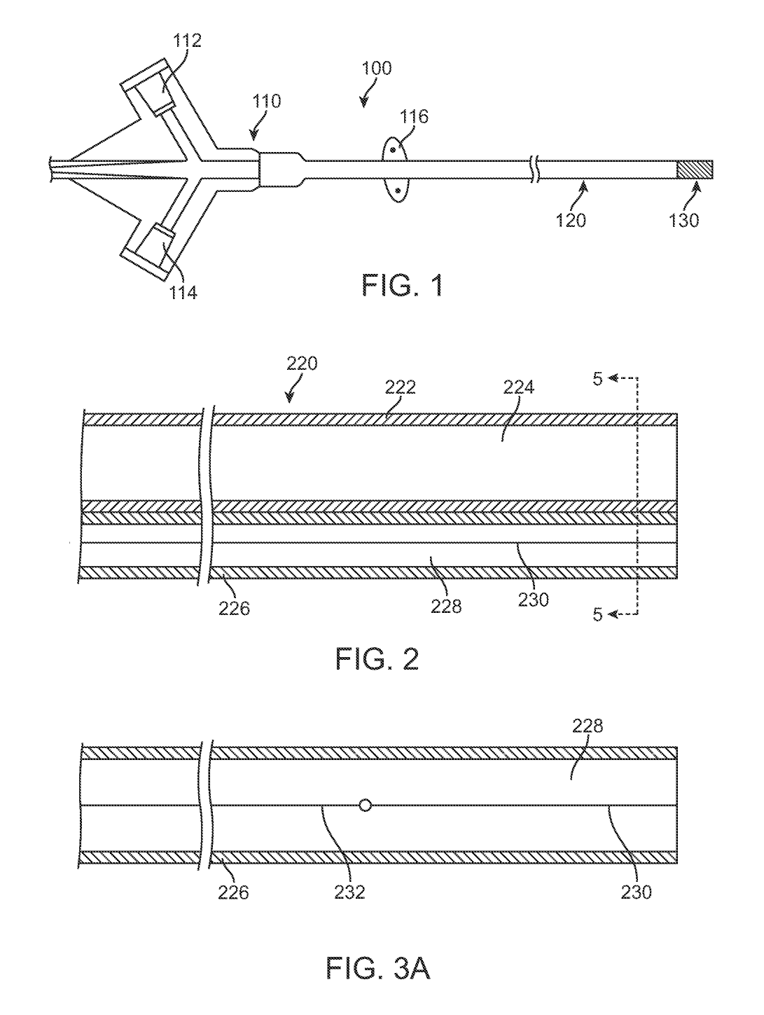

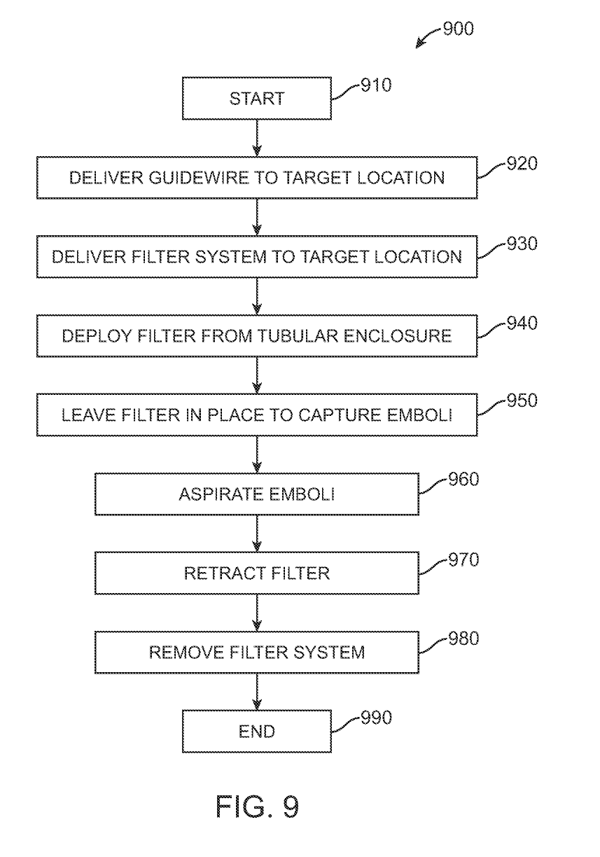

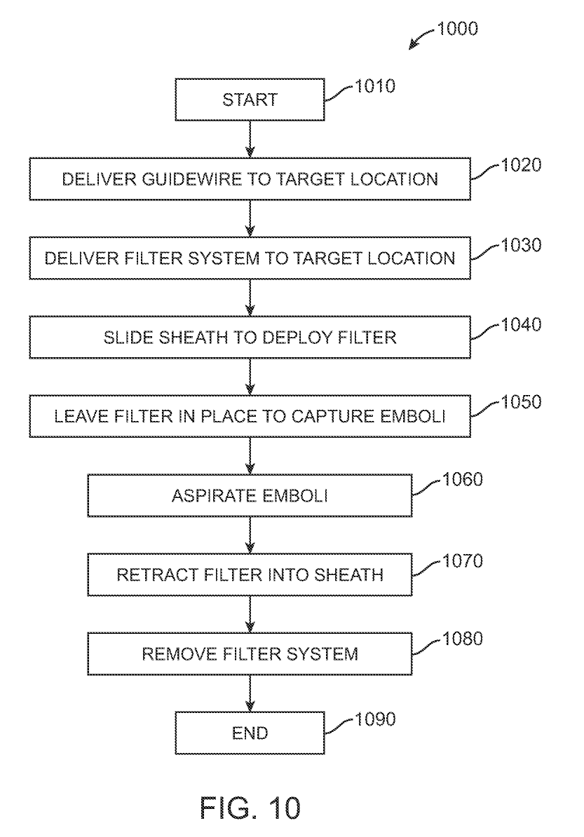

The principles and operation of devices and methods according to the present invention may be better understood with reference to the drawings and the accompanying description, where like reference numerals refer to like elements. More specifically, details of various IVC filter systems and components thereof will be described with reference to FIGS. 1-8. Details of various methods for using such systems will be described with reference to FIGS. 9-10.

With reference to FIG. 1 a typical IVC filter system 100 of the present invention is illustrated. The device includes a proximal portion 110, a distal portion 120 and a filter portion 130. Additionally an aspiration port 112 and an infusion port 114 are shown. In use, the proximal portion 110 remains outside the body of the patient. The aspiration port 112 and infusion port 114 may be operably connected to aspiration means and infusion means, respectively. Alternatively, the aspiration port 112 and infusion port 114 may be combined into one port.

FIG. 2 represents a cut-away view of one embodiment 220 of the distal portion of the device of FIG. 1. This embodiment includes an elongate element 222 and a tubular enclosure 226. Together the elongate element and tubular enclosure form an integral structure in which they are longitudinally adjacent one another. The elongate element 222 has an aspiration lumen 224 therethrough. The tubular enclosure has a lumen 228 which contains a filter 230 in an essentially straight delivery configuration.

While in FIG. 2 the tubular enclosure is illustrated as being adjacent to the elongate element other configurations may be utilized such as, for example, having the tubular enclosure concentric the elongate element. Together the elongate element and the tubular enclosure comprise an integral structure that extends from the distal end where the filter is deployed to the proximal end as shown in FIG. 1 as element 110. In a preferred embodiment this integral structure is a catheter. The catheter may have different degrees of flexibility along its length, for example, the proximal portion 110 may be relatively stiff and the distal portion 120 may be relatively flexible. The catheter may be constructed of various materials typically used for intravascular delivery catheters, for example, polymers such as high density polyethylene, low density polyethylene, polyurethanes, elastomeric polyamides, block polyamide/ethers, silicones, and copolymers thereof; or of one or more metallic materials such as stainless steel, tool steel, nickel-titanium alloy, Nitinol, nickel-chromium alloy, nickel-chromium-iron alloy, cobalt alloy, tungsten or tungsten alloys, MP35N, and the like; or a composite structure of one or more polymers in combination with one or more metals (e.g., a stainless steel braid over a polymer tube). The overall diameter of the catheter may range from about 2.0 mm to about 6.0 mm. The diameter of the aspiration lumen 224 may be from about 1.5 mm to about 5.5 mm. The diameter of the tubular enclosure lumen 228 may be between about 0.4 mm to about 2.2 mm.

The filter 230 is formed from a superelastic metallic wire having a conical coil shape when in its deployed configuration. A representation of such filter is shown in FIG. 4. In its delivery configuration, filter 230 is essentially straight. Preferred superelastic metals include shape memory metals such as Nitinol. The wire typically would have a diameter of between about 0.35 mm and 2.0 mm.

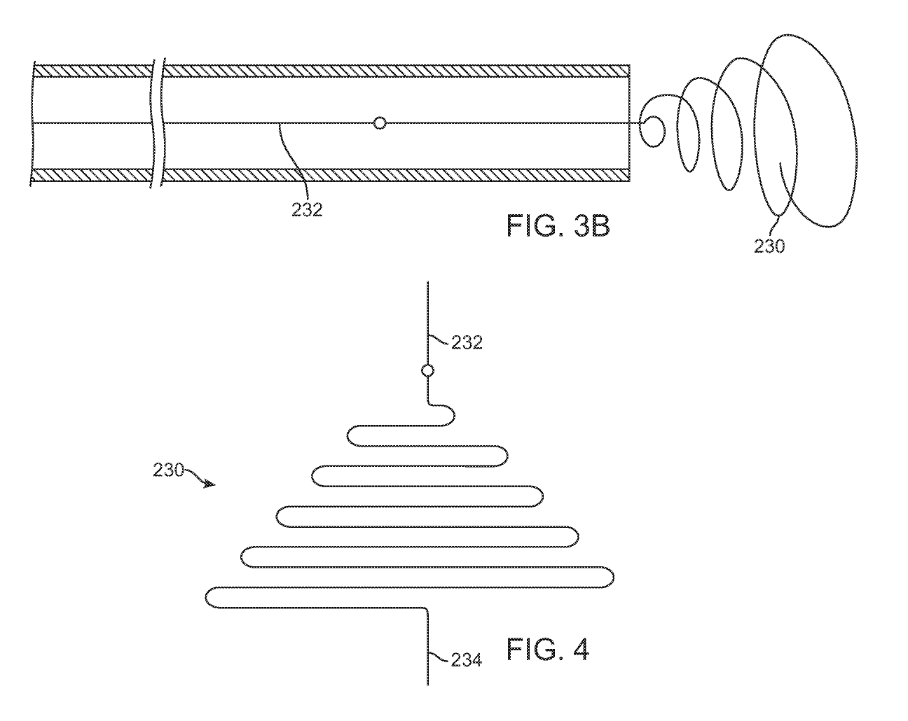

FIGS. 3A and 3B illustrate the tubular enclosure 226 and filter 230 of FIG. 2 in the delivery and deployed configurations, respectively. In order for the filter to be deployed and later retracted into the tubular enclosure, there must be a connection to the filter that extends through and outside the proximal portion 110 of the device. This connection, or introducer, 232 may be an extension of the filter wire itself or may be a separate element that is connected to the proximal portion of the filter.

In order to more easily load the coiled filter 230 into the proximal end of the tubular member lumen 228 whereby it needs to become an essentially straight wire, and to avoid the need for accessories to load the filter into the lumen, the filter may have a distal tip 234 as shown in FIG. 4. The distal tip 234 is essentially a straight extension of the coiled filter 230. By first advancing the distal tip into the proximal end of the tubular member lumen 228, the remainder of the filter follows and uncoils more easily.

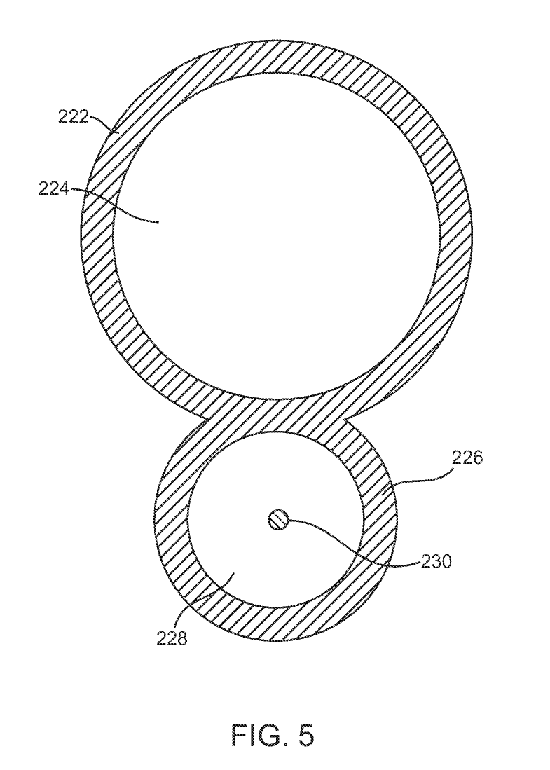

FIG. 5 illustrates a radial cross-section of the device of FIG. 2. As mentioned above, although the tubular enclosure 226 is shown as being outside the elongate member 222, in alternate embodiments it may be within the elongate member.

The aspiration lumen 224 may also be used as a guidewire lumen or there may be a separate guidewire lumen within the elongate member. The tubular enclosure lumen 228 may also be used as a guidewire lumen. In such case a guidewire in inserted into the tubular enclosure lumen, the device positioned at the target location, the guidewire withdrawn and replaced by the filter.

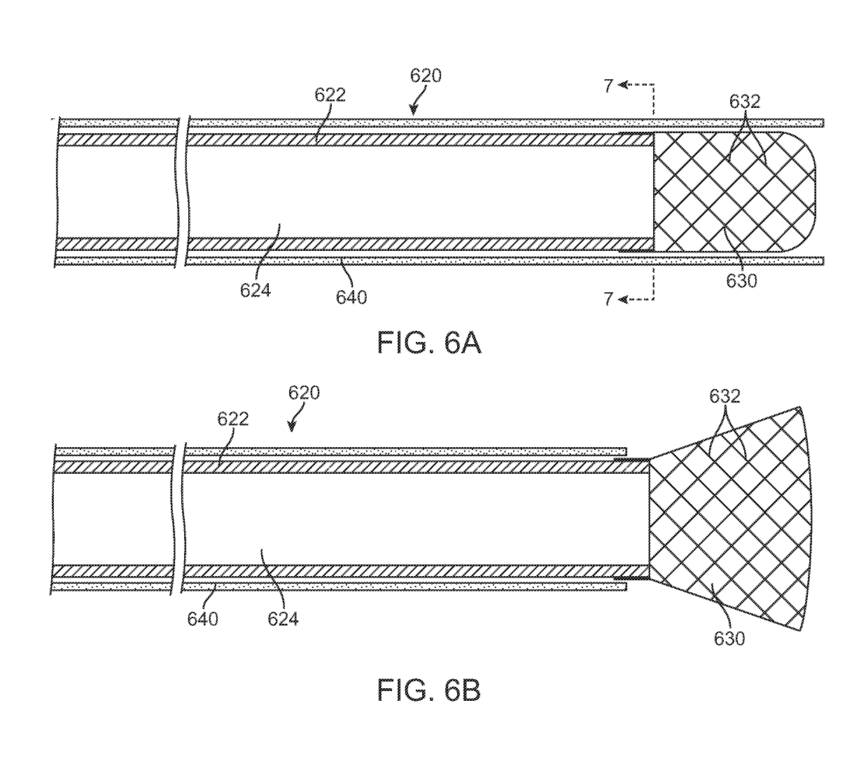

FIGS. 6A and 6B illustrate another embodiment of the device 100 and show an expanded view of distal portion 620, in the delivery position and the deployed position, respectively. In this embodiment the filter 630 is depicted as a mesh basket that in one embodiment is attached over the distal end of elongate member 622. The mesh basket of filter 630 is composed of a plurality of fibers 632 woven or otherwise assembled together to form the filter. It is understood that filter 630 may be attached to elongate element 622 in another manner, for example, with the elongate element disposed over the filter. A sheath 640 is slidably disposed over the elongate member and filter in FIG. 6A. In FIG. 6B the sheath 640 is shown as having been withdrawn, allowing the filter 630 to self-expand and assume a conical shape.

As mentioned with respect to the earlier embodiment, the elongate member is typically a delivery catheter and may be constructed in a manner typically utilized for intravascular delivery catheters as described above. The elongate member would continue from its distal end where the filter is attached to its proximal end as shown in 110 of FIG. 1. The elongate member 622 would typically have a diameter of between about 2.0 mm to about 6.0 mm and the aspiration lumen 624 would typically have a diameter of between about 1.5 mm to about 5.5 mm.

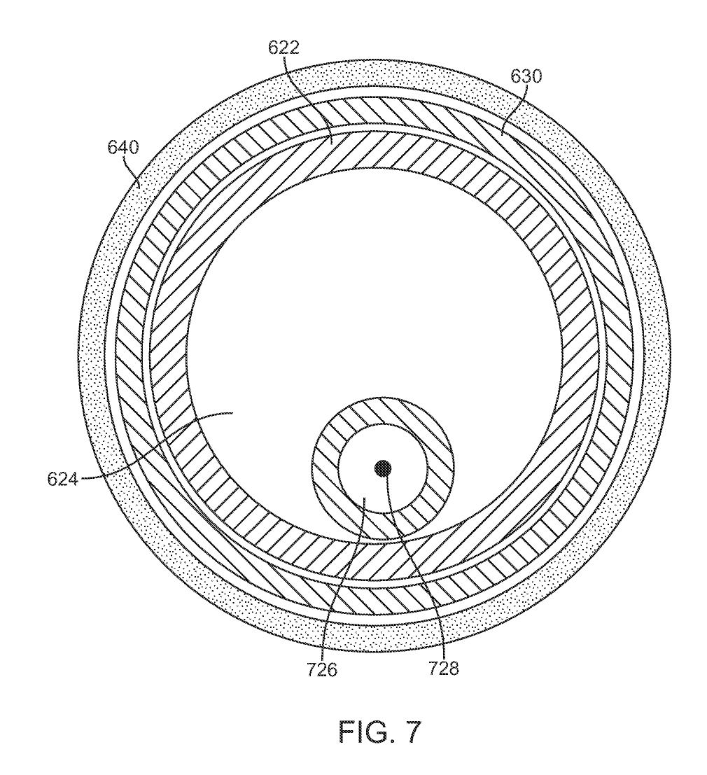

FIG. 7 illustrates a radial cross-section of the device of FIG. 6A, but additionally including a guidewire lumen 726 and a guidewire 728. Guidewire 728 may also be inserted directly in elongate member lumen 624 without the need for a separate guidewire lumen.

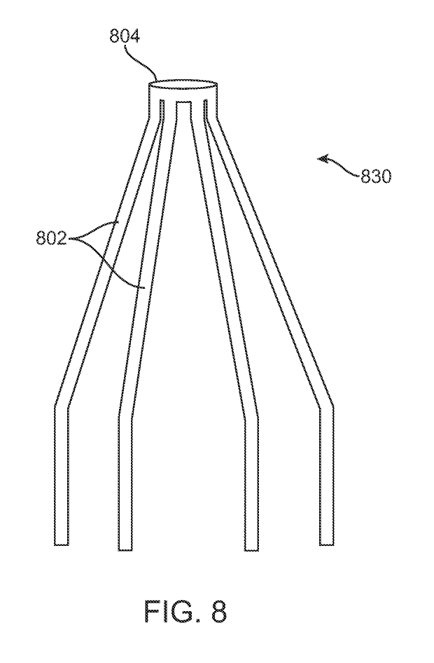

The filter mesh fibers 632 may be constructed from any number of materials such as solid or hollow polymeric fibers or solid or hollow metallic fibers such as stainless steel alloys, cobalt chromium alloys (e.g. MP35N, Conichrome®, Phynox® and Elgiloy®), nickel titanium alloys (e.g. Nitinol), etc. The fibers are preferably comprised of a superelastic material such as Nitinol or a spring temper stainless steel or cobalt chromium alloy. Alternatively, the fibers may be a composite, for example, a polymeric fiber having a wire coil wound within to provide greater structural integrity. Suitable polymers that may be used for such fibers include, for example, ePTFE, silicones, polyethylenes, high-density polyethylenes, low-density polyethylenes, polyimides, PEBAX, etc. The filter may also be a traditional slotted tube type stent design fabricated from Nitinol or other self-expanding, shape memory materials. As illustrated in FIGS. 6A and 6B the filter is a woven mesh. Alternatively, filter designs typically used for permanent filters, but without hooks, barbs or piercing means, may be utilized. Such designs, as illustrated in FIG. 8 typically include a series of legs 802 that are attached to an annular ring 804. The legs are biased outward and self-expand upon deployment. The legs 802 are comprised of solid or hollow tubes formed from polymeric fibers or metallic fibers as described above for filter 630.

The sheath 640 may be constructed from, for example, high density polyethylene, low density polyethylene, polyurethanes, elastomeric polyamides, block polyamide-ethers, silicones, and copolymers thereof; or of one or more metallic materials such as stainless steel, tool steel, nickel-titanium alloy, Nitinol, nickel-chromium alloy, nickel-chromium-iron alloy, cobalt alloy, tungsten or tungsten alloys, MP35HN, and the like; or a composite structure of one or more polymers in combination with one or more metals (e.g., a stainless steel braid over a polymer tube), and typically would have a lubricious coating on its interior and exterior surfaces to facilitate its slidability with respect to the elongate member, filter, guidewire and vasculature. The lubricious coating may be hydrophilic or hydrophobic, although hydrophilic lubricants are generally preferred. Suitable examples of hydrophilic lubricants, include, but are not limited to, polyalkylene glycols and alkoxy polyalkylene glycols; copolymers of methylvinyl ether and maleic acid; maleic anhydride polymers; polyalkylene oxides, particularly the polyethylene oxides; poly((meth)acrylic acids); polymers of hydroxyl-substituted lower alkyl(meth)acrylates, such 2-hydroxyalkyl(meth)acrylate; polyvinylalcohols, hydrophilic polyamides; poly(meth)acrylamides; poly(N-isopoly(meth)acrylamides); poly(sodium-4-styrenesulfonates) and poly(sodium vinylsulfonates); poly(3-hydroxybutyric acids); poly(N-vinyl lactams) such as the polyvinylpyrrolidones; hydrophilic polyurethanes; polyethyleneimines; poly(sodium(meth)acrylates); methyl cellulose, hydroxylmethyl cellulose, hydroxyethyl cellulose; polyvinylsulfonic acid; heparin; dextran and dextan sulfate and other modified dextrans; poly(saccharides); chondroitin sulphate; lecithin; and so forth, as well as copolymers thereof, and mixtures thereof. Hydrophobic lubricants include, but are not limited to, silicones (i.e. organosiloxane polymers), functionalized silicones, hydrolyzable silanes which form silicones, fluorosilanes and other fluoropolymers, cellulose esters and ethers, ethyl cellulose, cellulose nitrate, cellulose acetate, cellulose acetate butyrate, cellulose acetate propionate, hydrophobic polyurethanes, polyacrylates, natural and synthetic elastomers, polyacetals, hydrophobic polyamides, polyvinylidene chloride, polycarbonate, homopolymers and copolymers of vinyl compounds, polyvinylchloride, glycerin, olive, vegetable, and other natural oils, and so forth.

FIG. 9, represents a flowchart depicting the various steps in a method 900 of capturing and removing venous emboli from a patient.

Method 900 begins at step 910. At step 920 a guidewire is delivered to a target location in the inferior vena cava of a patient. Typically, the inferior vena cava is accessed intravascularly through one of a number of veins such as the femoral vein, the jugular vein or the subclavian vein. In the preferred embodiment of this method, the subclavian or jugular vein would be the access point through which the device would enter the body, allowing for the patient to be ambulatory following placement and securement of the catheter.

At step 930 an IVC filter system as set forth in FIGS. 2-6 is advanced over the guidewire to the target location. The guidewire may be inserted in the aspiration lumen 224, or a separate guidewire lumen may be utilized.

At step 940 the filter is delivered to the target location (deployed) by removing it from the distal end of the tubular enclosure 226. The removal may be performed by either pushing the filter out of the tubular enclosure using the introducer 232, or by pulling back on the catheter shaft comprising the tubular enclosure and elongate member while maintaining the position of the introducer, or by moving both the catheter shaft and introducer relative to one another. Once the filter 230 is free of the tubular enclosure it assumes its shape-memory conical coil shape as illustrated in FIGS. 3B and 4.

At step 950 the filter is left in place in the inferior vena cava for a sufficient period of time to capture any venous emboli that might be present and prevent them from migrating through the heart to the lungs. Typically, this period would range from a few days to a few weeks and would, in any event, extend beyond a time when the risk of further emboli has passed. In order that the filter not migrate from its implanted location, both the filter/introducer and the catheter (elongate element and tubular enclosure) need to be held in place relative to one another, and the catheter needs to be held in place relative to the patient. The former may be accomplished, for example, by providing a locking mechanism at the point the introducer 232 exits from the proximal portion 110 of the catheter. The latter may be accomplished, for example, by temporarily suturing or stapling the proximal portion of the catheter to the patient at the point the catheter enters the patient's body. This may be done, for example, by providing a tab 116, as shown in FIG. 1, that may be fixed or slidably mounted on the proximal portion of the catheter and can be tightened so that it is fixed to a desired location on the catheter. In this manner, the IVC filter system can be left in place during the entire process of capturing and removing emboli, thus eliminating the need for a second catheterization or other invasive procedure.

Optionally, an anticoagulant agent and/or a thrombolytic agent may be infused through the IVC filter system to the deployed filter 230 to wholly or partially dissolve or break up captured emboli, thus assisting in the removal of such emboli by subsequent aspiration. Such anticoagulant or thrombolytic agent may be infused through infusion port 114 and continuing through the aspiration lumen 224 to the filter 230. The infusion would involve the administration of a suitable pharmaceutically acceptable formulation of the anticoagulant or thrombolytic agent from a reservoir, either by hydraulic means or manually, for example, by means of a syringe. Optionally, there may be present a separate infusion lumen that is within or adjacent to the aspiration lumen. Suitable anticoagulant agents are well known in the art and include, for example, heparin, coumadin, aspirin, ticlopidine, clopidogrel and prasugrel. Suitable thrombolytic agents are well known in the art and include, for example, tPA, reteplase, alteplase, tenecteplase, activase, lanoteplase, staphylokinase, streptokinase and urokinase. The infusion port 114 would typically contain a valve fitting to prevent the backflow of venous blood.

At step 960 captured emboli are aspirated through the aspiration lumen and out of the patient through aspiration port 112. Aspiration port 112 would typically be operably connected to a vacuum source to provide aspiration capability. The aspiration port 112 would typically contain a valve fitting to prevent the backflow of venous blood when aspiration is not being performed.

At step 970 the filter 230 is retracted back into the tubular enclosure 226. This is accomplished by reversing the movements of the filter/introducer and catheter described in step 940. As the filter is retracted it uncoils and assumes an essentially straight wire shape.

At step 980 the entire IVC filter system 100 is withdrawn from the patient. Any sutures or similar fastening means for holding the filter system in place are undone to allow for free movement of the filter system.

Method 900 ends at step 990.

FIG. 10 represents a flowchart depicting the various steps in another method 1000 of capturing and removing venous emboli from a patient.

Method 1000 begins at step 1010. At step 1020 a guidewire is delivered to a target location in the inferior vena cava of a patient. Typically, the inferior vena cava is accessed intravascularly through one of a number of veins such as the femoral vein, the jugular vein or the subclavian vein. In the preferred embodiment of this method, the subclavian or jugular vein would be the access point through which the device would enter the body, allowing for the patient to be ambulatory following placement and securement of the catheter.

At step 1030 an IVC filter system as set forth in FIGS. 6 and 7 is advanced over the guidewire to the target location. The guidewire may be inserted in the aspiration lumen 624, or a separate guidewire lumen 726 may be utilized.

At step 1040 the filter is delivered to the target location (deployed) by sliding the sheath 640 in the proximal direction relative to the catheter, thus exposing the filter 630 or 830 and allowing it to assume its shape-memory configuration as illustrated in FIG. 6B.

At step 1050 the filter is left in place in the inferior vena cava for a sufficient period of time to capture any venous emboli that might be present and prevent them from migrating through the heart to the lungs. Typically, this period would range from a few days to a few weeks and would, in any event, extend beyond a time when the risk of further emboli has passed. In order that the filter not migrate from its implanted location, the catheter (elongate element) needs to be held in place relative to the patient. This may be accomplished, for example, by temporarily suturing the proximal portion of the catheter to the patient at the point the catheter enters the patient's body. This may be done, for example, by providing a tab 116, as shown in FIG. 1, which tab is slidably mounted on the proximal portion of the catheter and can be tightened so that it is fixed to a desired location on the catheter.

In order that the filter not migrate from its implanted location, the catheter needs to be held in place relative to the patient. This may be accomplished, for example, by temporarily suturing or stapling the proximal portion of the catheter to the patient at the point the catheter enters the patient's body. This may be done, for example, by providing a tab 116, as shown in FIG. 1, that may be fixed or slidably mounted on the proximal portion of the catheter and can be tightened so that it is fixed to a desired location on the catheter. In this manner, the IVC filter system can be left in place during the entire process of capturing and removing emboli, thus eliminating the need for a second catheterization or other invasive procedure.

Optionally, an anticoagulant agent and/or a thrombolytic agent may be infused through the IVC filter system to the deployed filter 630 or 830 to wholly or partially dissolve or break up captured emboli, thus assisting in the removal of such emboli by subsequent aspiration. Such anticoagulant or thrombolytic agent may be infused through infusion port 114 and aspiration lumen 624 to the filter 630 or 830. The infusion would involve the administration of a suitable pharmaceutically acceptable formulation of the anticoagulant or thrombolytic agent from a reservoir, either by hydraulic means or manually, for example, by means of a syringe. Optionally, there may be present a separate infusion lumen that is within or adjacent to the aspiration lumen. Suitable anticoagulant agents are well known in the art and include, for example, heparin, coumadin, aspirin, ticlopidine, clopidogrel and prasugrel. Suitable thrombolytic agents are well known in the art and include, for example, tPA, reteplase, alteplase, tenecteplase, activase, lanoteplase, staphylokinase, streptokinase and urokinase. The infusion port 114 would typically contain a valve fitting to prevent the backflow of venous blood.

At step 1060 captured emboli are aspirated through the aspiration lumen and out of the patient through aspiration port 112. Aspiration port 112 would typically be operably connected to a vacuum source to provide aspiration capability. The aspiration port 112 would typically contain a valve fitting to prevent the backflow of venous blood when aspiration is not being performed.

At step 1070 the filter 630 or 830 is retracted back into the sheath 640. This is accomplished by either retracting the catheter relative to the sheath, or sliding the sheath in a distal direction relative to the catheter, or both. As the filter is retracted it is compressed and assumes a configuration similar to that depicted in FIG. 6A.

At step 1080 the entire IVC filter system 100 is withdrawn from the patient. Any sutures or similar fastening means for holding the filter system in place are undone to allow for free movement of the filter system.

Method 1000 ends at step 1090.

Having thus described the several embodiments of the present invention, those of skill in the art will readily appreciate that other embodiments may be made and used that fall within the scope of the appended claims. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of this invention.

What is claimed is:

1. An inferior vena cava filter system comprising
a. an expandable filter having proximal and distal ends,
b. an elongate element having proximal and distal ends and an aspiration lumen therethrough, and
c. a tubular enclosure surrounding said filter.

2. The system of claim 1 wherein said elongate element and said tubular enclosure form an integral structure in which they are longitudinally adjacent to or concentric with each other.

3. The system of claim 2 wherein the proximal end of said elongate element further contains an aspiration port.

4. The system of claim 3 wherein the proximal end of said elongate element further contains an infusion port.

5. The system of claim 2 wherein the elongate element further contains a guidewire lumen.

6. The system of claim 2 wherein the filter has shape memory characteristics such that it comprises a delivery configuration and a deployed configuration.

7. The system of claim 6 wherein the filter is composed of a superelastic material.

8. The system of claim 7 wherein the superelastic material is Nitinol.

9. The system of claim 6 wherein the filter in the delivery configuration is essentially straight.

10. The system of claim 6 wherein the filter in the deployed configuration has a conical coil shape.

11. The system of claim 6 wherein the filter further comprises an introducer, said introducer being attached at the proximal end of said filter.

12. The system of claim 11 wherein said introducer is an extension of said filter.

13. The system of claim 1 wherein the proximal end of said filter is attached to the distal end of said elongate element.

14. The system of claim 1 wherein said tubular enclosure is a sheath that is slidably disposed around said elongate element and filter.

15. The system of claim 13 wherein the proximal end of said elongate element further contains an aspiration port.

16. The system of claim 13 wherein the proximal end of said elongate element further contains an infusion port.

17. The system of claim 13 wherein the elongate element further contains a guidewire lumen.

18. A method of capturing and removing venous emboli comprising
a. delivering a guidewire to a target location in the inferior vena cava of a patient having a body,
b. delivering an inferior vena cava filter system over the guidewire to the target location, the system comprising
i. a filter having proximal and distal ends,
ii. an elongate element having proximal and distal ends and an aspiration lumen therethrough, and
iii. a tubular enclosure surrounding said filter,
wherein the proximal end of said elongate element further contains an aspiration port, wherein the filter comprises a shape memory metallic wire that is essentially straight when within the tubular enclosure and that has a conical coil configuration when deployed,
c. delivering the filter to the target location, thereby allowing the filter to assume its shape memory conical coil configuration,
d. leaving the filter in place for a determined period of time to capture emboli,
e. aspirating captured emboli through the aspiration lumen,
f. retracting the filter into the tubular enclosure, and
g. removing the system from the patient.

19. The method of claim 18 further comprising, after step (d), the infusion of an anticoagulant agent or a thrombolytic agent through an infusion port.

20. The method of claim 19 wherein the anticoagulant agent is selected from the group consisting of heparin, coumadin, aspirin, ticlopidine, clopidogrel and prasugrel, and the thrombolytic agent is selected from the group consisting of tPA, reteplase, alteplase, tenecteplase, activase, lanoteplase, staphylokinase, streptokinase and urokinase.

21. The method of claim 18 further comprising, after step (c), securing the proximal end of the elongate member to the outside of the patient's body.

22. The method of claim 21 wherein the securing comprises suturing or stapling.

23. A method of capturing and removing venous emboli comprising
a. delivering a guidewire to a target location in the inferior vena cava of a patient having a body,
b. delivering an inferior vena cava filter system over the guidewire to the target location, the system comprising
i. a filter having proximal and distal ends,
ii. an elongate element having proximal and distal ends and an aspiration lumen therethrough, and
iii. a tubular enclosure surrounding said filter,
wherein the proximal end of said filter is attached to the distal end of said elongate element and said tubular enclosure is a sheath that is slidably disposed around said elongate element and filter,
c. sliding said sheath with respect to said elongate element to deploy the filter,
d. leaving the filter in place for a determined period of time to capture emboli,
e. aspirating captured emboli through the aspiration lumen,
f. retracting the filter into the sheath, and
g. removing the system from the patient.

24. The method of claim 23 further comprising, after step (d), the infusion of an anticoagulant agent or a thrombolytic agent through an infusion port.

25. The method of claim 20 wherein the anticoagulant agent is selected from the group consisting of heparin, coumadin, aspirin, ticlopidine, clopidogrel and prasugrel and the thrombolytic agent is selected from the group consisting of tPA, reteplase, alteplase, tenecteplase, activase, lanoteplase, staphylokinase, streptokinase and urokinase.

26. The method of claim 23 further comprising, after step (c), securing the proximal end of the elongate member to the outside of the patient's body.

27. The method of claim 26 wherein the securing comprises suturing or stapling.
